(19) United States
(12) Patent Application Publication
(10) Pub. No.: US 2010/0151449 A1
(43) Pub. Date: Jun. 17, 2010

(54) Method for poduction of l-amino acids by fermentation

(76) Inventors: Jan Marinhagen, Julich (DE); Lothar Eggeling, Julich (DE); Hermann Sahm, Julich (DE)

Correspondence Address: KF ROSS PC, 5683 RIVERDALE AVENUE, SUITE 203 BOX 900, BRONX, NY, 10471-0900, US

(21) Appl. No.: 11/919,325

(22) PCT Filed: Apr. 20, 2006
(86) PCT No.: PCT/DE2006/000685

§ 371 Date: Oct. 25, 2007

(30) Foreign Application Priority Data
Apr. 29, 2005 (DE) 102005019967.4

(51) Int. Cl. C12Q 1/68; C12P 13/04; C07H 21/00; C07H 21/04; C12N 15/74; C12N 1/21; C12N 9/10

(52) U.S. Cl. 435/6; 435/106; 536/23.1; 536/23.2; 435/320.1; 536/23.7; 435/252.32; 435/193

(57) ABSTRACT

The invention relates to a method for production of L-amino acids by fermentation. According to the invention, the activity of the alanine transaminase is ether reduced or inhibited, whereby in particular the amino acids L-valine, L-lysine and L-isoleucine are produced with increased yield. Furthermore, the nucleic acids according to seq. No. 1 from position 101 to 1414 are identified as the sequence coding for the alanine transaminase gene. Use of the above permits the production of L-alanine.

Object of the invention is a method of producing L-amino acids.

L-amino acids are used in human medicine, in the pharmaceutical industry, the food industry and in animal nutrition.

It is known that amino acids are produced by the fermentation of strains of coryneform bacteria, particularly Corynebacterium glutamicum. Due to their high importance, their production processes undergo continuous improvement. Improvements of the method can relate to fermentation measures, such as stirring and introducing oxygen, or the composition of the culture media, such as the sugar concentration during fermentation, or the processing into a product form by ion exchange chromatography, for example, or the intrinsic performance properties of the microorganism per se.

To improve the performance properties of these microorganisms, mutagenesis, selection and mutant selection methods are employed. In this way, strains are obtained that are resistant to antimetabolites or auxotrophic for metabolites having regulatory relevance and produce L-amino acids. For example, U.S. Pat. No. 5,521,074 describes a Corynebacterium strain that is resistant to L-valine and sensitive to fluoropyruvate. Furthermore, EP 0287123 [U.S. Pat. No. 5,188,948] describes that Corynebacteria resistant to mycophenolic acid can advantageously be used for producing L-valine. It is also known that mutants with mutated valyl-tRNA synthetase, combined with additional mutations, can be used for L-valine production (EP 0519113 A1, U.S. Pat. No. 5,658,766).

Recombinant DNA technology is used additionally to improve the intrinsic properties of L-amino acid-producing strains of Corynebacterium. It has been described, for example, that the increased expression of the biosynthesis genes ilvBN, ilvC, ilvD can be advantageously used for L-valine production (EP 1155139 B1, EP 0356739 B1). It is furthermore known that the reduction or elimination of the threonine-dehydratase gene ilvA and/or of genes involved in the synthesis of pantothenate can be used for L-valine production (EP 1155139 B1).

Improved performance properties are also achieved by a decrease in by-product formation. This is accomplished in part by a suitable fermentation process. It is also known that the formation of by-products can be reduced by the expression of special genes. For example, the expression of avtA results in the increased formation of L-leucine and the decreased formation of the accompanying amino acid L-valine (WO 00171021 A1 [U.S. Pat. No. 7,202,060]). Furthermore, it is known that frequently by-products can be formed from pyruvate, such as lactate and alanine that are both produced directly from pyruvate (Uy D. et al, J. Biotechnol. 2003 Sep. 4; 104 (1-3): 173-184). On the other hand, pyruvate is required as a building block for amino acids, such as L-isoleucine, L-valine and L-lysine. Due to the intrinsic properties of the microorganism, the methods according to the state of the art may still result in the formation of alanine as an undesirable by-product, and relatively low yields of the desired amino acids, such as L-valine, are achieved.

It is therefore the object of the invention to provide new ways for the improved production of L-amino acids by fermentation, the acids preferably being formed from pyruvate and resulting in higher product yields. In particular, the yield should be increased for the production of L-valine, L-isoleucine and L-lysine.

Surprisingly, the goal is attained in that the activity of the alanine transaminase relative to the naturally occurring strain is reduced or completely deactivated, or that the alanine production is reduced. Furthermore, the goal is attained in that an alanine transaminase is identified.

Alanine transaminase according to the invention in particular means L-alanine transaminase.

Surprisingly, the yield for the production of amino acids, particularly L-valine, L-lysine and L-isoleucine, can be considerably increased.

The invention will be explained hereinafter in detail. The FIGS. show plasmids:

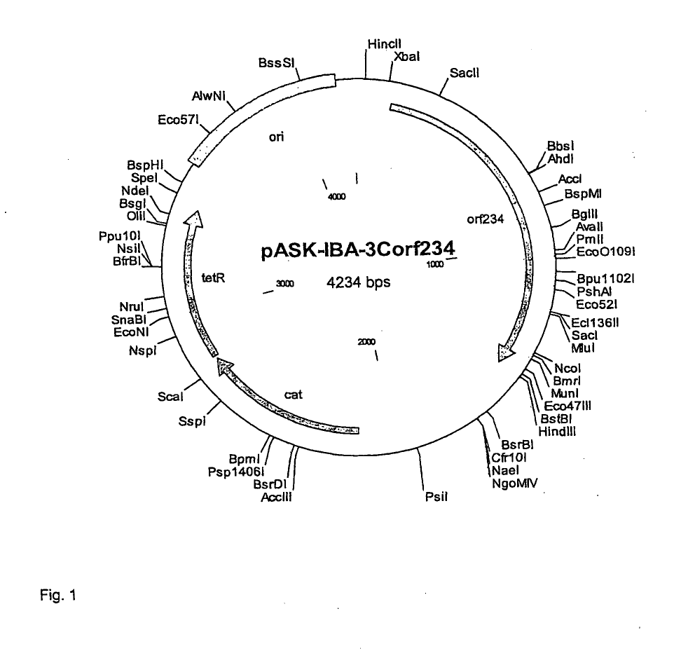

FIG. 1 is a plasmid for detecting the activity of the alanine transaminase gene according to example 3.

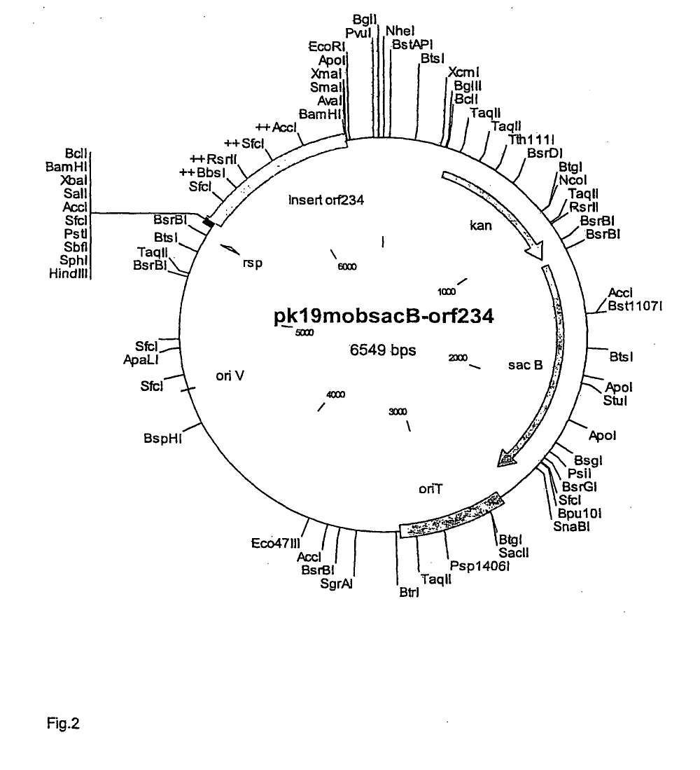

FIG. 2 is a plasmid that is used for the deletion of the alanine transaminase gene according to example 4.

Also shown are:

Sequence listing 1: A gene sequence coding for alanine transaminase.

Sequence listing 2: The amino acid sequence of alanine transaminase.

Sequence listing 1 codes from nucleotides 101 to 1414 for alanine transaminase.

Sequence listing 2 shows the sequence of alanine transaminase coded by the nucleotides 101 to 1414 of sequence listing 1.

According to the invention, for example the following measures can be taken:

The elimination of the alanine transaminase gene can be brought about by deletion or disruption.

For this purpose, surprisingly gene number NCg12757 deposited in the publicly accessible database of the National Institute of Health as identified as coding for alanine transaminase, the function of the gene being previously unknown. The gene is therefore likewise an object of the invention. The sequence is listed in sequence listing 1 (nucleotides 101-1414).

The invention also includes gene structures that comprise the sequence according to sequence listing 1.

These may be chromosomes, plasmids, vectors, phages, viruses and the like. Furthermore, the gene sequence or nucleotide sequence is also part of the invention.

The sequence according to sequence listing 1 (nucleotides 101-1414) codes for alanine transaminase and can therefore be used for production of the same. For this purpose, the person skilled in the art can employ known methods, for example overexpression, increase of promoters or starting codons. By way of example, the organisms disclosed in this application can be used for the production of alanine transaminase.

Furthermore, every gene coding for alanine transaminase can be deleted or subjected to disruption.

To decrease the activity of alanine transaminase, for example the following methods can be employed:
mutation of the sequence coding for the alanine transaminase gene, and/or
reduction of the expression of alanine transaminase and/or
reduction or elimination of the promoter preceding the alanine transaminase and/or
mutation or deletion of the starting codon preceding the alanine transaminase gene and/or
blockage of the catalytic center of alanine transaminase, for example by adding substances blocking this center, or chemical substitution, for example caused by mutation.

According to the invention, it is preferable if coryneform bacteria, particularly Corynebacterium glutamicum, are used.

The object of the invention is also a plasmid that is used for the deletion or deactivation of the gene cording for alanine transaminase. The plasmid comprises internal sequences of the alanine transaminase gene or sequences adjacent to the 3′ and 5′ ends of the alanine transaminase gene.

It is essential that the plasmid comprises sequences of the alanine transaminase gene or those that are adjacent to the alanine transaminase gene, preferably the areas that are directly adjacent to the 3′ and 5′ ends of the gene and ideally the plasmid shown in FIG. 2.

The amino acid L-valine is used in human medicine, in the pharmaceutical industry, the food industry and in animal nutrition.

The object of the invention is also a microorganism or a transformed cell or a recombinant cell, wherein the production of alanine is reduced or completely deactivated or wherein the activity of alanine transaminase is reduced or completely deactivated.

The strains used produce L-valine or L-isoleucine or L-lysine preferably already before deletion of the alanine transaminase gene.

Preferred embodiments are described in the claims.

The term “modification” in this context describes the reduction of the intracellular activity of one or more biosynthesis enzymes to produce amino acids (proteins) in a microorganism, the amino acids being coded by the appropriate DNA in that, for example, a weak promoter or a gene or allele is used that codes for a corresponding enzyme with reduced activity or expresses the appropriate gene (protein) to a reduced extent and optionally combines these measures, or in that the gene is even completely deleted.

The microorganisms that are the object of the present invention can produce L-valine or also other L-amino acids that are formed from pyruvate, from glucose, saccharose, lactose, fructose, maltose, molasses, starch, cellulose or from glycerin and ethanol. These are representatives of coryneform bacteria, particularly of the Corynebacterium species. Among the Corynebacterium species particularly the Corynebacterium glutamicum type should be mentioned that is known in the expert world for its ability to produce L-amino acids.

Suitable starting strains of the Corynebacterium species, particularly of the Corynebacterium glutamicum type, are for example the known wild type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869 and

Brevibacterium divaricatum ATCC14020 and mutants made from these that produce an excess of L-amino acids or strains that have been modified according to the invention.

It was found that following the reduction or deactivation of the gene coding for alanine transaminase coryneform bacteria exhibit improved production of the L-amino acids of valine, leucine and isoleucine.

The nucleotide sequence of the alanine transaminase gene is automatically known from the development of the complete genome sequence of C. glutamicum (Kalinowski et al, 2003, J. Biotechnol., 104:5-25; Ikeda M., and Nakagawa S. 2003 Appl. Microbiol. Biotechnol. 62:99-109), however without the association of an open reading frame for alanine transaminase being known. The open reading frame identified and described hereinafter by way of example, this frame codes for alanine transaminase carrying the number NCg12747 and being stored in the publicly accessible database of the National Institute of Health (http://www.ncbi.nlm.nih.gov) and also under Cg12844 in the publicly accessible Data bank of Japan (http://gib.genes.nig.ac.ip).

The alanine transaminase gene described by these numbers is preferred according to the invention as a starting point for the invention. Furthermore, alleles of the alanine transaminase gene can be used that are obtained, for example, from the degeneracy of the genetic code or by functionally neutral sense mutations or by deletion or insertion of nucleotides.

So as to achieve a weakening effect, either the expression of the alanine transaminase gene or the catalytic properties of the enzyme protein can be reduced. Likewise, the catalytic property of the enzyme protein can be modified with respect to the substrate specificity thereof. Optionally, the two measures can be combined.

The weakening of the gene expression can be performed by suitable culture management or genetic modification (mutation) of the signal structures of the gene expression. Signal structures of the gene expression are, for example, repressor genes, activator genes, operators, promoters, attenuators, ribosome binding sites, the starting codon and terminators. The person skilled in the art can find relevant information, for example, in the patent application WO 96/15246 [U.S. Pat. No. 5,965,391], in Boyd and Murphy (J. Bacteriol. 1988. 170: 5949), in Voskuil and Chambliss (Nucleic Acids Res. 1998 26: 3548, in Jensen and Hammer (Biotechnol. Bioeng. 1998 58: 191), in Patek et al (Microbiology 1996 142: 1297 and in well-known genetics and molecular biology textbooks, such as the textbook by Knippers (Molekulare Genetik (Molecular Genetics), 8^{th} edition, Georg Thieme publishing house, Stuttgart, Germany, 2001) or that of Winnacker (Gene and Kione (Genes and Clones), VCH publishing house, Weinheim, Germany, 1990) .

Mutations resulting in a modification of the catalytic properties of enzyme proteins, particularly in a modified substrate specificity, are known from the state of the art. Examples that should be mentioned are the publications by Yano et al 1998 Proc. Natl. Acad. Sci. USA, 95:5511-5, Oue S. et al J. Biol. Chem. 1999, 274:2344-9 and Onuffer et al Protein Sci. 1995 4:1750-7. Possible mutations are transitions, transversions, insertions and deletions as well as methods of directed evolution. Instructions for producing such mutations and proteins are part of the state of the art and are described in well-known textbooks (R. Knippers Molekulare Genetik (Molecular Genetics), 8^{th} edition, 2001, Georg Thieme publishing house, Stuttgart, Germany), or overview articles (N. Pokala 2001, J. Struct. Biol. 134:269-81; A. Tramontano 2004, Appl. Chem. Int. Ed Engl. 43:3222-3; N. V. Dokholyan 2004, Proteins. 54:622-8; J. Pei 2003, Proc. Natl. Acad. Sci. USA. 100:11361-6; H. Lilie 2003, EMBO Rep. 4:346-51; R. Jaenicke Appl. Chem. Int. Ed. Engl. 42:140-2).

The weakened expression of the genes or mutated genes occurs in accordance with conventional methods of gene replacement in that the native chromosomal gene is replaced with the mutated gene, as is described, for example, in Morbach et al (Appl. Microbiol. Biotechnol. 1996, 45:612-620). The deletion of the gene is performed as described by Scharzer and Pühler (Biotechnology 1990, 9: 84-87), and in Schafer et al (Appl. Environ. Microbio. 1994, 60: 756-759). The transformation of the desired strain with the vector for gene replacement or for deletion occurs by means of conjugation or electroporation of the starting strain. The conjugation method is described, for example, in Schäfer et al (Appl. Environ. Microbio. 1994, 60: 756-759). Methods for transformation are described, for example, in Tauch et al (FEMS Microbiological Letters (1994)123:343-347).

In this way, the alanine transaminase gene can be deleted or the allele thereof can be changed into C. glutamicum.

Furthermore, in addition to weakening or deleting the alanine transaminase activity, it may be advantageous for the production of L-valine to strengthen, particularly overexpress, one or more of the genes selected from the group consisting of
the ilvBN genes coding for acetohydroxy acid synthase,
the ilvC gene coding for isomeroreductase,
the ilvD gene coding for dehydratase,
the ilvE gene coding for transaminase C,
or to strengthen or overexpress alleles of these genes, particularly
the ilvBN genes coding for feedback-resistant acetohydroxy acid synthase.

Furthermore, in addition to reducing the alanine transaminase activity, it may be advantageous for the production of L-valine to deactivate or reduce one or more of the genes selected from the group consisting of
the panBCD genes coding for pantothenate synthesis,
the lipAB genes coding for lipoic acid synthesis,
the aceE, aceF, ipD genes coding for pyruvate dehydrogenase,
the genes for [sic] the ATP synthase A subunit, ATP synthase B subunit, ATP synthase C subunit, ATP synthase alpha subunit, ATP synthase gamma subunit, ATP synthase subunit [sic], ATP synthase epsilon subunit, ATP synthase delta subunit.

Finally, in addition to the weakening of alanine transaminase, it may be advantageous for the production of L-valine to deactivate undesirable side reactions that produce leucine, for example (Nakayama: “Breeding of Amino Acid Producing Micro-organisms,” in: Overproduction of Microbial Products, Krumphanzl, Sikyta, Vanek (eds.)/Academic Press, London, UK, 1982).

The microorganisms produced according to the invention can be cultivated continuously or discontinuously in a batch process (batch cultivation) or a fed-batch process or repeated fed-batch process for the purpose of valine production. A summary of known cultivation methods is described in the textbook by Chmiel (Bioprozesstechnik 1. Einführung in die Bioverfahrenstechnik (Bioprocess Technology 1. Introduction into Bioprocess Technology) (Gustav Fischer publishing house, Stuttgart, 1991)) or in the textbook by Storhas (Bioreaktoren and periphere Einrichtungen (Bioprocess reactors and peripheral Devices) (Vieweg publishing house, Braunschweig/Wiesbaden, 1994)).

The culture medium to be used must be matched with the requirements of the respective microorganisms. Descriptions of culture media for various microorganisms are outlined in the handbook “Manual of Methods for General Bacteriology” by the American Society for Bacteriology (Washington D.C., USA, 1981). Possible carbon sources are sugars and carbohydrates, such as glucose, saccharose, lactose, fructose, maltose, molasses, starch and cellulose, oils and fats, such as soy bean oil, sunflower oil, peanut oil and coconut fat, fatty acids, such as palmitic acid, stearic acid and linolic acid, alcohols, such as glycerin and ethanol, and organic acids, such as acetic acid. These substances can be used individually or as mixtures. Nitrogen sources can be organic, nitrogen-containing compounds such as peptones, yeast extract, meat extract, malt extract, corn rock water, soy bean flour and urea, or inorganic compounds, such as ammonium sulfate, ammonium chloride, ammonium phosphate, ammonium carbonate and ammonium nitrate. The nitrogen sources can be used individually or as mixtures. Possible phosphorus sources are potassium dihydrogen phosphate or dipotassium hydrogen phosphate or the corresponding sodium-containing salts. The culture medium must furthermore comprise salts of metals, such as magnesium sulfate or ferric sulfate that are necessary for growth. Finally, essential growth substances, such as amino acids and vitamins, can be used in addition to the above substances. The above-mentioned substances can be added to the culture in the form of a single batch or may be suitably fed during cultivation.

For pH control of the culture, alkaline compounds are suitably used, such as sodium hydroxide, potassium hydroxide or ammonia, or acid compounds, such as phosphoric acid or sulfuric acid. To control the foam development, anti-foaming agents such as fatty acid polyglycol esters may be used. So as to maintain the stability of plasmids, selectively acting substances, such as antibiotics, may be added to the medium. In order to maintain aerobic conditions, oxygen or oxygen-containing gas mixtures, such as air, are introduced in the culture. The temperature of the culture is generally 20° C. to 45° C., preferably 25° C. to 40° C. The culture is continued until maximum L-valine has formed. This objective is typically achieved within 10 to 160 hours.

### EXAMPLES

### Example 1

### Cloning Alanine Transaminase

With the help of the PCR reaction, a DNA fragment was amplified that comprises the alanine transaminase gene. The following primers were used:

orf234-for:
5′ - ATGGTA (GGTCTC) AAATGACTACAGACAAGCGCAAAACC
T - 3′
orf234rev:
5′ - ATGGTA (GGTCTC) AGCGCTCTGCTTGTAAGTGGACAGGAA
G - 3′

The primers listed were synthesized by MWG Biotech AG (Anzinger Str. 7a, D-85560 Ebersberg), and the PCR reaction was performed in accordance with standard protocols (Innis et al PCR Protocols. A guide to Methods and Applications. 1990. Academic Press). With the primers, a DNA fragment of approximately 1.3 kb was obtained that codes for alanine transaminase. The primers additionally comprise the interface of the restriction enzyme Bsal that are provided in the above nucleotide sequences in parentheses.

The amplified DNA fragment of approximately 1.3 kb was identified in 0.8% agarose gel and isolated from the gel with existing methods (QIAquik Gel Extraction Kit, Quiagen, Hilden). The ligation of the fragment was performed with the SureCloning Kit (Amersham, UK) in the pASK-IBA-3C expression vector (IBA, Göttingen). The ligation batch was used to transform E. coli DH5 (Grant et al 1990. Proceedings of the National of Sciences of the United States of America USA, 87:4645-4649). The selection of strains comprising plasmid was performed by plating the transformation batch on LB plates comprising 25 mg per liter of chloramphenicol.

Following plasmid isolation, the resulting plasmids were characterized by restriction digest and gel electrophoretic analysis. The plasmid was named pASK-IBA-3Corf234. It is illustrated in FIG. 1.

### Example 2

### Isolation of Alanine Transaminase

E. coli DH5 with pASK-IBA-3Corf234 was used at 30° C. in 100 ml LB with 25 mg per liter of chloramphenicol until an optical density of 0.5 was reached. Then 0.01 ml of an anhydrotetracycline solution was added that comprised 2 mg of anhydrotetracycline per milliliter of dimethyl formamide. The culture was incubated for 3 hours at 30° C. Then, the cells were harvested for 12 minutes at 4° C. and 5000 revolutions per minute by means of centrifugation. Thereafter, the cell pellets were resuspended in washing buffer (100 mM trihydroxymethyl amino methane, 1 mM ethylene diamine tetraacetic acid, pH 8) and transferred into an Eppendorf reaction vessel. The cell disruption was performed at 0° C. by means of an ultrasonic cell disruptor (Branson Sonifier W-250, Branson Sonic Power Company, Danbury, USA; sound application duration 10 minutes, pulse length 20%, sound application intensity 2). Following the ultrasound treatment, the cell debris was separated by centrifugation (30 minutes, 13,000 rpm, 4° C.) and obtained as a raw extract in the form of supernatant.

In order to isolate the protein, StrepTactin affinity columns from the manufacturer IBA (IBA, Göttingen, Germany) were filled with 1 ml bed volume of StrepTactin sepharose. Following the equilibration of the columns with washing buffer from the manufacturer IBA, 1 ml of the raw extract was placed on the sepharose. After the extract had run through, the affinity column was rinsed five times with 1 ml washing buffer each. The elution of the alanine transaminase protein was performed with elution buffer comprising 100 mM Tris, 1 mM EDTA and 2.5 mM desthiobiotin, pH 8. The elution fractions were aliquoted, frozen at −20° C. and used directly for the enzyme test.

### Example 3

### Determining the Activity of Alanine Transaminase

The reaction batch of the enzyme test had a total volume of 1 ml: 0.2 ml 0.25 M Tris/HCl, pH 8, 0.005 ml alanine transaminase protein and 0.1 ml 2.5 mM pyridoxal phosphate, as well as 0.1 ml 40 mM pyruvate and 0.1 ml 0.5 M L-glutamate, or 0.1 ml 40 mM pyruvate and 0.1 ml 0.5 M aspartate, or 0.1 ml 40 mM pyruvate and 0.1 ml 0.5 M a-amino-butyrate, or 0.1 ml 40 mM pyruvate and 0.1 ml 0.5 M L-glutamate without alanine transaminase protein. The enzyme test was carried out at 30° C. in a thermocycler 5436 from the company Eppendorf (Hamburg). The reaction was started by adding the protein. The addition of 30 ml of a stop reagent (6.7% (v/v) perchloric acid (70%), 40% (v/v) ethanol (95%) in water) to 50 ml of the test batch stopped the enzyme test. So as to prepare the samples for the detection of the produced amino acids by reversed-phase HPLC, 20 ml of a neutralization buffer (20 mM Tris, 2.3 M dipotassium carbonate, pH 8) was added. The precipitation deposited due to the neutralization of the perchloric acid was is centrifuged off (13,000 rpm, 10 minutes) and the supernatant was used in various dilutions for quantification by means of HPLC. This occurred following automatic derivatization with o-phthaldialdehyde as described (Hara et al 1985, Analytica Chimica Acta 172: 167-173). As Table 1 shows, the isolated protein catalyzes the amination dependent on L-glutamate, L-aspartate and a-aminobutyrate from pyruvate into alanine.

TABLE 1

| | | | | Specific |
|---|---|---|---|---|
| Protein | Amino donator | Amino acceptor | Product | affinity |
| Alanine | L-glutamate | Pyruvate | Alanine | 26.6 |
| transaminase | | | | |
| Alanine | L-aspartate | Pyruvate | Alanine | 3.0 |
| transaminase | | | | |
| Alanine | a-amino | Pyruvate | Alanine | 8.4 |
| transaminase | butyrate | | | |
| Control | L-glutamate | Pyruvate | Alanine | 0.0 |

The specific affinity is provided in micromole of product per minute and milligram of alanine transaminase protein.

### Example 4

### Deletion of the Alanine Transaminase Gene

With the help of the PCR reaction, two DNA fragments were amplified that flank the alanine transaminase gene. The following primers were used:

Del234_1:
5′ - CG (GGATCC) CATGCAACCGATCTGGTTTTGTG - 3′
Del234_2:
5′ - CCCATCCACTAAACTTAAACAGCGCTTGTCTGTAGTCACCC
G - 3′
Del234_3:
5′ - TGTTTAAGTTTAGTGGATGGGCGCCTGGGTAACTTCCTGTC
C - 3′
Del234_4:
5′ - CG (GGATCC) GATTGATCATGTCGAGGAAAGCC - 3′

The primers listed were synthesized by MWG Biotech AG (Anzinger Str. 7a, D-85560 Ebersberg), and the PCR reaction was performed in accordance with standard protocols (Innis et al PCR Protocols. A guide to Methods and Applications. 1990. Academic Press). The primers were used to amplify two DNA fragments of approximately 400 by each, which flank the gene for alanine transaminase. The primers De1234_{—}1 and De1234_{—}4 additionally comprise the interface of the restriction enzyme BamHI that is provided in the above nucleotide sequences in parentheses. The amplified DNA fragments of approximately 400 kb were identified in 0.8% agarose gel and isolated from the gel with existing methods (QIAquik Gel Extraction Kit, Quiagen, Hilden). With the help of a second PCR reaction, during which the two previously amplified DNA fragments were used as template DNA (Link et al, 1997, J. Bacteriol. 179:6228-6237), a fragment of approximately 800 by was amplified. This fragment comprises both DNA areas flanking the alanine transaminase. The amplified DNA fragment of approximately 800 kb was identified in 0.8% agarose gel and isolated from the gel with existing methods (QIAquik Gel Extraction Kit, Quiagen, Hilden).

The ligation of the fragment was performed with the SureCloning Kit (Amersham, UK) in the pkl9mobsacB deletion vector (Schäfer et al, 1994, Gene (Genes) 145:69-73) The ligation batch was used to transform E. coli DH5 (Grant et al 1990. Proceedings of the National of Sciences of the United States of America USA, 87:4645-4649). The selection of strains comprising plasmid was performed by plating the transformation batch on LB plates comprising 50 mg per liter of kanamycin.

Following the plasmid isolation, the resulting plasmids were characterized by restriction digest and gel electrophoretic analysis. The plasmid was named pk19mobsacB-orf234. It is illustrated in FIG. 2.

The pk19mobsacB-orf234 plasmid was used for the transformation of the 13032DpanBC strain for kanamycin resistance. The strain is described in EP1155139 B1, and the transformation method is described in Kirchner et al, J. Biotechnol. 2003, 104:287-99.

The deletion of the gene for alanine transaminase was carried out in accordance with the protocol for the deletion of genes in Corynebacterium glutamicum according to Schäfer et al, 1994, Gene (Genes) 145:69-73 by two consecutive homologous recombinations.

With the help of the PCR reaction, the deletion of the gene for alanine transaminase was confirmed. The following primers were used:

Ko_delorf234_for: 5′ - CTGGGTATTCGCCACGGACGT - 3′
Ko_delorf234_rev: 5′ - TCGGCGGTGTCAAAAGCATTGC - 3′

The primers listed were synthesized by MWG Biotech AG, and the PCR reaction was performed in accordance with standard protocols (Innis et al PCR Protocols. A guide to Methods and Applications. 1990. Academic Press). The amplification of a 1 kB large DNA fragment confirmed the deletion of the gene for alanine transaminase. The resulting strain was named the 13032DpanBCDalaT strain.

### Example 5

### Reduction of the Alanine Formation and Increase of the L-Valine Formation through the Deletion of Alanine Transaminase

The 13032DpanBCDalaT strain as well as the 13032DpanBC control strain were used in the medium CGIII (Henkel et al, 1989, Appl. Environ. Microbiol. 55:684-8) at 30° C. Then the CGXII medium with an optical density of 1 was inoculated. The CGXII medium comprises the following per liter of substance: 20 g (NH₄)₂SO₄, 5 g urea, 1 g KH₂PO₄, 1 g K₂HPO₄, 0.25 g Mg₂O₄*7H₂O, 42 g 3-morpholinopropane sulfonic acid, 10 mg CaCl₂, 10 mg FeSO₄*7H₂O, 10 mg MnSO₄*H₂O, 1 mg ZnSO₄*7H₂O, 0.2 mg CuSO₄, 0.02 mg NiCl₂*6H₂O, 0.2 mg biotin, 40 g glucose, 0.5 mM pantothenate and 0.03 mg protocatechuic acid. The culture was incubated at 30° C. and 120 revolutions per minute and after 56 hours the alanine and L-valine accumulations in the medium were determined by means of HPLC. This occurred with o-phthaldialdehyde as described (Hara et al 1985, Analytica Chimica Acta 172: 167-173). The alanine and L-valine concentrations that were determined are listed in Table 2.

TABLE 2

| Strain | Alanine | L-valine |
|---|---|---|
| 13032ΔpanBC | 56.0 mM | 13.1 mM |
| 13032ΔpanBC | 44.1 mM | 20.9 mM |
| ΔalaT | | |

1. A method of the microbial production of L-amino acids wherein the alanine transaminase activity is reduced or deactivated or that the production of alanine is reduced or deactivated.

2. The method according to claim 1 wherein the alanine transaminase gene is deleted or subjected to disruption.

3. The method according to claim 1 wherein the sequence coding for alanine transaminase is mutated.

4. The method according to claim 1 wherein the expression of alanine transaminase is reduced or deactivated.

5. The method according to claim 1 wherein the promoter preceding the alanine transaminase gene is weakened or deactivated or replaced with a weaker promoter.

6. The method according to claim 1 wherein the starting codon preceding the alanine transaminase gene is weakened or deactivated with respect to its activity or replaced with a weaker starting codon.

7. The method according to claim 1 wherein the catalytic center of alanine transaminase is blocked.

8. The method according to claim 1 wherein at least one component of the group of genes consisting of
the ilvBN gene coding for acetyhydroxy acid synthase,
the ilvC gene coding for isomeroreductase,
the ilvD gene coding for dehydratase,
the ilvE gene coding for transaminase, or
the ilvBN gene coding for feedback-resistant acetohydroxy acid synthase
is strengthened or overexpressed.

9. The method according to claim 1 wherein at least one component of the group consisting of
the panBCD genes coding for pantothenate synthesis,
the lipAB genes coding for liponic acid synthesis,
the aceE, aceF, lpD genes coding for pyruvate dehyrogenase,
the genes for [sic] the ATP synthase A subunit, ATP synthase B subunit, ATP synthase C subunit, ATP synthase alpha subunit, ATP synthase gamma subunit, ATP synthase subunit [sic], ATP synthase epsilon subunit, ATP synthase delta subunit
are deactivated or reduced with respect to their activity.

10. The method according to claim 1 wherein coryneform bacteria are used.

11. The method according to claim 10 wherein bacteria of the Corynebacterium glutamicum species are used.

12. The method according to claim 10 wherein an organism from the group consisting of
Corynebacterium glutamicum ATCC13032
Corynebacterium acetoglutamicum ATCC15806
Corynebacterium acetoacidophilum ATCC13870
Corynebacterium thermoaminogenes FERM BP-1539
Brevibacterium flavum ATCC14067
Brevibacterium lactofermentum ATCC13869 and
Brevibacterium divaricatum ATCC14020 is used.

13. The method according to claim 1 wherein L-valine, L-isoleucine or L-lysine is produced.

14. A nucleotide sequence according to sequence 1, nucleotides 101 to 1414.

15. A gene structure comprising an alanine transaminase gene according to sequence 1, nucleotides 101 to 1414.

16. The gene structure according to claim 15 wherein it is mutated.

17. The gene structure according to claim 16 wherein it is mutated by insertion and/or deletion and/or replacement of nucleic acids.

18. A vector comprising a gene structure itself comprising an analyine transmine gene according to sequence 1, nucleotides 101 to 1414 or a nucleotide sequence according to sequence 1, nucleotides 101 to 1414.

19. The vector according to claim 18 wherein the vector is a plasmid, a phage or a virus.

20. A chromosome comprising a gene structure itself comprising an analyine transmine gene according to sequence 1, nucleotides 101 to 1414 or a nucleotide sequence according to sequence 1, nucleotides 101 to 1414.

21. The chromosome according to claim 20 wherein the alanine transaminase gene is partially or completely deleted.

22. A recombinant cell, comprising a gene structure according to itself comprising an analyine transmine gene according to sequence 1, nucleotides 101 to 1414 and/or
a vector comprising a gene structure itself comprising an analyine transmine gene according to sequence 1, nucleotides 101 to 1414 or a nucleotide sequence according to sequence 1, nucleotides 101 to 1414 and/or
a chromosome comprising a gene structure itself comprising an analyine transmine gene according to sequence 1, nucleotides 101 to 1414 or a nucleotide sequence according to sequence 1, nucleotides 101 to 1414 or
the nucleotide sequence according to sequence 1, nucleotides 101 to 1414.

23. The recombinant cell according to claim 22 wherein it has already produced L-lysine, L-valine or L-isoleucine prior to the modifications according to claim 16.

24. The recombinant cell according to claim 22 wherein it is a Corynebacterium.

25. The recombinant cell according to claim 24 wherein it is a Corynebacterium glutamicum.

26. The recombinant cell according to claim 25 wherein it is an organism from the group consisting of
Corynebacterium glutamicum ATCC13032
Corynebacterium acetoglutamicum ATCC15806
Corynebacterium acetoacidophilum ATCC13870
Corynebacterium thermoaminogenes FERM BP-1539
Brevibacterium flavum ATCC14067
Brevibacterium lactofermentum ATCC13869 and
Brevibacterium divaricatum ATCC14020.

27. Use of the gene structure comprising an alanine transaminase gene according to sequence 1, nucleotides 101 to 1414 or of a nucleotide sequence according to sequence 1, nucleotides 101 to 1414 to produce alanine.

28. A plasmid, comprising internal sequences of the alanine transaminase gene or the sequences adjacent to the 3′ and 5′ ends of the alanine transaminase gene.

29. Alanine transaminase, characterized by sequence 2.


SEQUENCE LISTING (10 sequences)

SEQ ID NO: 1 - DNA, 1414 bp, Corynebacterium glutamicum
gatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgctattcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtag

SEQ ID NO: 2 - protein, 437 aa, Corynebacterium glutamicum
MTTDKRKTSKTTDTANKAVGADQAARPTRRTTRRIFDQSEKMKDVLYEIRGPVAAEAERMELDGHNILKLNTGNPAVFGFDAPDVIMRDMIANLPTSQGYSTSKGIIPARRAVVTRYEVVPGFPHFDVDDVFLGNGVSELITMTTQALLNDGDEVLIPAPDYPLWTAATSLAGGKPVHYLCDEEDDWNPSIEDIKSKISEKTKAIVVINPNNPTGAVYPRRVLEQIVEIAREHDLLILADEIYDRILYDDAEHISLATLAPDLLCITYNGLSKAYRVAGYRAGWMVLTGPKQYARGFIEGLELLAGTRLCPNVPAQHAIQVALGGRQSIYDLTGEHGRLLEQRNMAWTKLNEIPGVSCVKPMGALYAFPKLDPNVYEIHDDTQLMLDLLRAEKILMVQGTGFNWPHHDHFRVVTLPWASQLENAIERLGNFLSTYKQ

SEQ ID NO: 3 - DNA, 39 bp, Artificial Sequence
  Description of Artificial Sequence Synthetic Construct
atggtaggtctcaaatgactacagacaagcgcaaaacct

SEQ ID NO: 4 - DNA, 40 bp, Artificial Sequence
  Description of Artificial Sequence Synthetic Construct
atggtaggtctcagcgctctgcttgtaagtggacaggaag

SEQ ID NO: 5 - DNA, 31 bp, Artificial Sequence
  Description of Artificial Sequence Synthetic Construct
cgggatcccatgcaaccgatcgttgtttgtg

SEQ ID NO: 6 - DNA, 42 bp, Artificial Sequence
  Description of Artificial Sequence Synthetic Construct
cccatccactaaacttaaacagcgcttgtctgtagtcacccg

SEQ ID NO: 7 - DNA, 42 bp, Artificial Sequence
  Description of Artificial Sequence Synthetic Construct
tgtttaagtttagtggatgggcgcctgggtaacttcctgtcc

SEQ ID NO: 8 - DNA, 31 bp, Artificial Sequence
  Description of Artificial Sequence Synthetic Construct
cgggatccgattgatcatgtcgaggaaagcc

SEQ ID NO: 9 - DNA, 21 bp, Artificial Sequence
  Description of Artificial Sequence Synthetic Construct
ctgggtattcgccacggacgt

SEQ ID NO: 10 - DNA, 22 bp, Artificial Sequence
  Description of Artificial Sequence Synthetic Construct
tcggcggtgtcaaaagcattgc